(19) United States
(12) Patent Application Publication
(10) Pub. No.: US 2006/0014698 A1
(43) Pub. Date: Jan. 19, 2006

(54) Nebulized pharmaceutical compositions for the treatment of bronchial disorders

(76) Inventors: Michael F. O'Connor, Wilmette, IL (US); Edward T. Naureckas, Oak Park, IL (US); Imre Noth, Chicago, IL (US)

Correspondence Address: LAHIVE & COCKFIELD, LLP., 28 STATE STREET, BOSTON, MA, 02109, US

(21) Appl. No.: 11/182,279

(22) Filed: Jul. 14, 2005

Related U.S. Application Data
(60) Provisional application No. 60/588,154, filed on Jul. 14, 2004.

(51) Int. Cl. A61K 38/05; A61K 31/737

(52) U.S. Cl. 514/18; 514/54

(57) ABSTRACT

The present invention provides methods and pharmaceutical compositions for the treatment of bronchial disorders by inhalation therapy. In various embodiments, the active ingredient of the pharmaceutical compositions is one or more of a sulfated pentasaccharide and a peptidomemtic of a tripeptide. In various embodiments, the active ingredient of the pharmaceutical compositions is one or more of argatroban and fondaparinux.

### CROSS-REFERENCE TO RELATED APPLICATIONS

The present application claims the benefit of U.S. Provisional Patent Application No. 60/588,154, filed Jul. 14, 2004, the entire contents of which are incorporated herein by reference.

### BACKGROUND

Asthma, a chronic disease characterized by airway hyperactivity and bronchoconstriction, occurs in 5-8% of the U.S. population and is an extraordinarily common cause of pulmonary impairment. Asthma is characterized by airway inflammation, hyperresponsiveness, bronchial smooth muscle contraction and, in some cases, airway smooth muscle hyperplasia. Despite considerable research efforts, the precise cellular and molecular mechanisms that induce airway hyperactivity in asthmatics remain unknown. Studies have shown, however, that airway inflammation is important in stimulating airway smooth muscle contraction that results in bronchial constriction. Mononuclear cells recruited into the airway are stimulated to release inflammatory mediators. Release of these newly formed mediators as well as eicosonoids induces edema of the bronchial submucosa by increasing vascular permeability. This increase in vascular permeability results in the capillary leakage of serum proteins, which activate thrombin.

Thrombin is increasingly recognized as an important mediator of inflammation in a variety of pathophysiologic processes. A variety of modulators of thrombin activity, including activated recombinant proteins and anti-thrombin have been investigated as therapies for thrombin/coagulation system mediated inflammatory processes, with recombinant Protein C now being widely used as a therapy for sepsis (a syndrome of uncontrolled systemic inflammation in the body usually driven by an infection). A variety of basic science and clinical investigations have demonstrated a possible role for platelet activation in the pathogenesis of asthma, and in the use of thrombin inhibitors for treating it (for further information, refer to the Kaneoyoshi, Roth, Frou, and the Ahmed References, and U.S. Pat. No. 6,355,626).

Although a wide variety of thrombin modulating compounds are used in medicine, they are not thought of as interchangeable across applications. For example, argatroban is widely used in patients with antibodies to heparin, and is more active at clot bound thrombin than heparin/antithrombin complexes are, but is not commonly used as a therapy for pulmonary embolism. Indeed, there is at present little or no discussion of argatroban's use in this regard except as an alternative to heparin in these patients. Another example of the generally narrow use of anti-thrombotic and anticoagulant agents in medicine is in the treatment of crescendo/unstable angina. A variety of newer anti-coagulant agents (Abciximab/Reopro and Tirofiban/Aggrastat), active at proteins on the surface of platelets (IIB/IIIa) are commonly used in this setting, but not in any other situation in medicine.

One factor that prevents thrombin modulating compounds from being thought of as interchangeable across applications is that the clinical properties of these compounds are not self-evident from their chemical structures, even to those skilled in the art. For example, the direct and indirect thrombin inhibiting effects of heparin have been intensively studied. The development of the low molecular weight heparins demonstrated that chemical modification of heparins (naturally occurring and unfractionated) changes their thrombin inhibiting activities in ways that are not predictable with the current art. Consequently, the anti-coagulant activity of low molecular weight heparins cannot be measured using the same laboratory tests used to measure the anti-coagulant activity of unfractionated heparin.

Compound antigenicity is another factor that prevents thrombin modulating compounds from being thought of as interchangeable across applications. For example, all heparins are known to be antigenic. This is a consequence of several factors, including the fact that they are glycosolated proteins (which are antigenic) their large size, and the fact that most are derived from animal sources, and hence are proteins which do not naturally occur in man. In addition to containing foreign proteins, animal derived heparins are glycosylated in ways that may not be found in man and which may also be antigenic. Antibodies to heparins are a well described problem in medicine.

Another factor to be taken into consideration in using a compound across medical applications is the ability to provide an effective means of compound delivery for the treatment application. For example, in the treatment of bronchospasm and bronchial inflammation conditions the ability to deliver effective compounds via nebulization is an important consideration. However, whether a compound will be effective via nebulization and how to produce an effective nebulized compound is not predictable. For example, a variety of compounds have proven to be effective via nebulization in ways that those familiar with the art did not foresee. One impressive example is the nebulization of tobramycin, a high molecular weight aminoglycoside antibiotic, to treat pseudomonas pneumonia in patients with cystic fibrosis.

However, despite the finding that activation of thrombin may play a role in bronchial constriction, there remains a need for pharmaceutical compositions to treat bronchospasm and bronchial inflammation.

### SUMMARY OF THE INVENTION

The present invention relates to methods and pharmaceutical compositions for intratracheobronchial administration to treat bronchial disorders. Specifically, the present invention relates to atomized, nebulized, aerosolized or dry powder inhalation (e.g., of micronized active ingredient) pharmaceutical compositions containing a thrombin modulating compound for the treatment of one or more of asthma, bronchospasm, bronchial constriction, bronchial inflammation, and bronchial hypertrophy.

In various aspects, the present invention provides pharmaceutical compositions containing a thrombin modulating compound for the treatment of bronchial disorders via inhalation therapy.

In various aspects, the present invention provides methods for treatment of a bronchial disorder by inhalation therapy with a pharmaceutical composition containing a thrombin modulating compound.

In various aspects, the invention comprises using a pharmaceutical composition containing a thrombin modulating compound in the manufacture of a medicament for the treatment of a bronchial disorder.

Preferably, in all aspects of the invention, the thrombin modulating compound is not a protein, i.e., it is a non-protein thrombin modulating compound. In various embodiments, the non-protein thrombin modulating compound comprises a pentasaccharide, and in various versions of these embodiments, a sulfated pentasaccharide. In various embodiments, the non-protein thrombin modulating compound comprises a peptidomemetic. In various versions of these embodiments, the peptidomemetic is of a tripeptide, is an azapeptide, or both. In various embodiments, the non-protein thrombin modulating compound comprises one or more of argatroban, efegatran, inogatran, napsagatran and fondaparinux.

In various embodiments of the methods and pharmaceutical compositions of the present invention, fondaparinux, argatroban, and related compounds can provide an effective treatment for bronchial spasm and bronchial constriction. In some patients, fondaparinux, and related compounds can be administered as a prophylactic to prevent one or more of bronchospasm, bronchial inflammation, and bronchial constriction, or to reduce the incidence or severity of one or more of them. In various embodiments of the methods and pharmaceutical compositions of the present invention, fondaparinux and related compounds can be effective at preventing airway smooth muscle and epithelial hypertrophy and cellular proliferation.

In various embodiments of the methods and pharmaceutical compositions of the present invention, fondaparinux, argatroban, and related compounds can provide an effective treatment of the local bronchial inflammation, associated bronchospasm, and bronchial constriction that occurs in asthmatics, patients with emphysema, bronchiectasis, bronchitis, and other states.

In various embodiments of the methods and pharmaceutical compositions of the present invention, argatroban, fondaparinux and related compounds can provide effective maintenance anti-inflammatory agents in the treatment of patients with bronchial inflammation and chronic bronchial constriction. In various embodiments of the methods and pharmaceutical compositions of the present invention, argatroban, fondaparinux and related compounds can be effective in the treatment of acute exacerbations of either (or both) bronchial inflammation and bronchospasm.

In various embodiments of the methods and pharmaceutical compositions of the present invention, fondaparinux, argatroban, and related compounds can be effective at preventing the long term changes associated with chronic bronchial inflammation and bronchospasm, including airway smooth muscle hypertrophy, epithelial hypertrophy, and inflammation mediated destruction such as bronchiectasis, ectasia of large and small airways, and diminished elastic recoil. Compounds described in U.S. Pat. No. 6,271,215 have this property and are representative of a class of compounds that can be formulated (e.g., nebulized, dry powder) for inhalation therapy of asthma and bronchial disorders. All of the compounds described in U.S. Pat. No. 6,271,215 are incorporated herein by reference.

For example, sulfated oligosaccharides, where the oligosaccharide has the general formula I:
R₁—(Rₓ)ₙ—R₂ (I)
where R₁ and R₂ and each Rₓ represent a monosaccharide unit, all of which may be the same or different, adjacent monosaccharide units being linked by 1→2, 1→3, 1→4 and/or 1→6 glycosidic bonds; and n is an integer of from 1 to 6, and preferably 3 or 4. The monosaccharide units which are linked together to form the oligosaccharides can be, e.g., a hexose, hexuronic, hexosamine or N-acetylhexosamine. A hexose can be, e.g., a furanose, such as, e.g., fructose; or a pyranose, such as, e.g., glucose, mannose, altrose, allose, talose, galactose, idose, and gulose. The hexoses can be in the D- and/or the L-configuration. The oligosaccharides of general formulae I can include compounds wherein the monosaccharide units are derivatised, e.g., where the units are phosphate, acetyl or other ester derivatives of monosaccharides.

Studies have demonstrated that inhalation of heparin is an effective treatment for exercise and allergy induced asthma, and that efficacy of inhaled pharmaceutical compositions increases as molecular weight decreases (Ahmed, Reference Nos. 1 and 2). Panettieri et al. in U.S. Pat. No. 6,355,626 describe examples of compounds used to treat asthma, these compounds are incorporated herein by reference. We have determined that the compounds described in U.S. Pat. No. 6,355,626 are suitable for delivery through intratracheobronchial administration. Accordingly, in various aspects, the present invention provides atomized, nebulized, aerosolized or dry powder inhalation (e.g., of micronized active ingredient) pharmaceutical compositions for the treatment of bronchospasm and bronchial inflammation containing an asthma treatment compound described in U.S. Pat. No. 6,355,626.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 schematically depicts a chemical structure of argatroban.

FIG. 2 schematically depicts a chemical structure of fondaparinux.

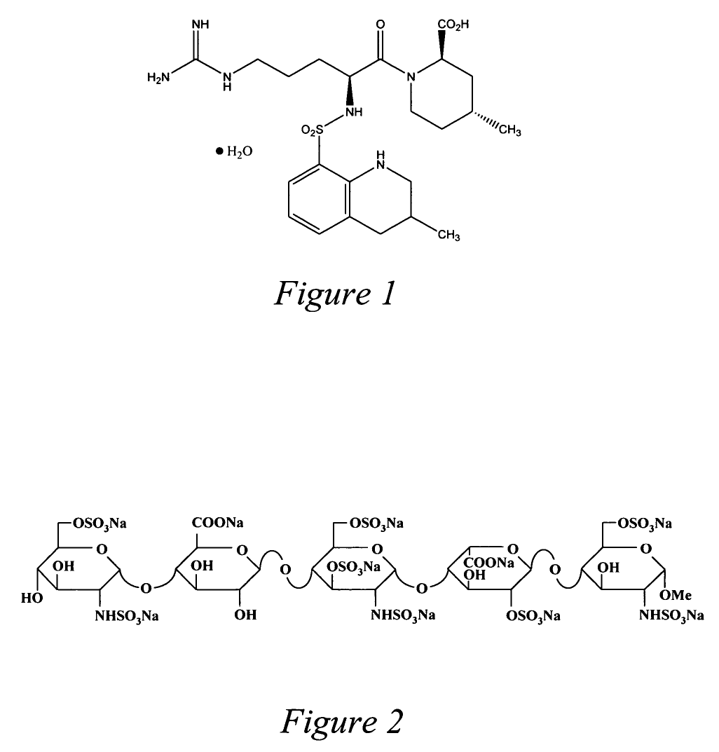

### DETAILED DESCRIPTION OF THE INVENTION

Prior to further describing the invention, it may be helpful to an understanding thereof to set forth definitions of certain terms to be used herein.

As used herein, the terms “active compound” and “active ingredient” are used interchangeably herein and refer to one or more thrombin modulating compounds and pharmaceutically acceptable salts thereof.

As used herein, the term “bronchial disorder” refers to the symptoms, disorders and/or conditions of or associated with one or more of asthma, bronchospasm, bronchial constriction, bronchial inflammation, and bronchial hypertrophy.

As used herein, the term “bronchial spasm” means an involuntary spasm of the breathing tubes of a patient. Bronchial constriction is both a term and a medical condition which is interchangeable with “bronchial spasm” in its use with respect to the purposes of this application.

As used herein, the term “bronchial inflammation” refers to an inflammation of the breathing tubes of a patient.

As used herein, the terms “effective amount”, “effective dose” and “effective dosage are used interchangeably herein and refer to an amount of a thrombin modulating compound sufficient to do one or more of the following: (1) inhibit or at least partially inhibit, either directly, indirectly, or both, an alpha-thrombin-induced effect in the breathing tubes; (2) produce a “therapeutic effect”, that is, reduce a sign or a symptom associated with a bronchial disorder; or (3) both inhibit or at least partially inhibit, either directly, indirectly, or both, an alpha-thrombin-induced effect in the breathing tubes and reduce a sign or a symptom associated with a bronchial disorder.

As used herein, the term “inhalation therapy” refers to all means of delivering a pharmaceutical compositions of the present invention to the respiratory tract during routine or assisted respiration (e.g., by intratracheobronchial, pulmonary, and/or nasal administration), including, but not limited to, delivery using atomization, nebulization and/or aerosolization of the composition.

As used herein, by the terms “inhibit”, “inhibiting” and “inhibition” it is meant that there is a decrease from the normal level of response of the breathing tubes to alpha-thrombin.

As used herein, “micronized” refers to particles (e.g., of an active ingredient, carrier, diluent, etc.) having a mean particle size that is greater than about 0.5 microns but less than about 25 microns. A micronized particle can be prepared by any acceptable method including, but not limited to, milling, and precipitation.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for administration by inhalation (e.g., by intratracheobronchial administration). The active compound can be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The terms “thrombin modulating compound” and “anti-thrombin agent” are used interchangeably herein and refer to any compound (including, but not limited to, pharmaceutically acceptable salts thereof) which deters the normal activities evoked by the presence of thrombin. Examples include all non-heparin non-protein direct and indirect thrombin inhibitors, but are not limited to, thrombin antagonists such as fondaparinux (indirect), argatroban (direct), NAPAP, Ro-466240, BM14.1248, L373-102-001u, and ximelagatran.

The term “treatment” as used herein, refers to the application or administration of a pharmaceutical composition of the present invention to a patient, who has a bronchial disorder, a symptom of a bronchial disorder, or a predisposition toward a bronchial disorder, with the purpose to cure, heal, alleviate, relieve, alter, remedy, ameliorate, improve or affect the bronchial disorder, the symptoms of the bronchial disorder or the predisposition toward the bronchial disorder. A treatment having a therapeutic effect can be one wherein a beneficial therapeutic response is generated in a patient (e.g., in the context of the prevention or treatment of a bronchospasm, bronchial constriction, bronchial inflammation, and/or bronchial hypertrophy).

As used hereinafter the term “medium chain fatty acids” refers to chains of alkyl groups terminating in a —COOH group and having 6-12 carbon atoms, preferably 8-10 carbon atoms. The term “short chain fatty acids” refers to chains of alkyl groups terminating in a —COOH group and having 4-8 carbon atoms. The term “alcohol” includes C₁- C₃ alcohols, such as methanol, ethanol and isopropanol.

The pharmaceutical compositions of the present invention include those suitable for intratracheobronchial, pulmonary, and/or nasal administration for the treatment of a bronchial disorder. Preferably, the pharmaceutical compositions of the present invention are formulated for delivery by atomization, nebulization, aerosolization and/or dry powder using, for example, an inhaler. The pharmaceutical compositions can be presented in unit dosage form and may be prepared by any methods known in the art of pharmacy.

The pharmaceutical compositions of the present invention comprise one or more thrombin modulating compounds as active ingredients. Where the active ingredient forms a suspension, the particle size is preferably relatively uniform, with substantially all the particles ranging between about 0.1 to about 25 microns in size, preferably between about 0.5 to about 10 microns in size, and more preferably between about 1 to about 5 microns in size. Particles larger than 25 microns may be held up in the oropharyngeal cavity, while particles smaller than about 0.5 micron preferably are not utilized, since they would be more likely to be exhaled and, therefore, not reach the lungs of the patient.

Preferably, the thrombin modulating compound is not a protein. Examples of such non-protein thrombin modulating compounds for use in the pharmaceutical compositions of the invention include, pentasaccharides and peptidomemetics. Preferred pentasaccarides include sulfated pentasaccarides, such as, for example, fondaparinux. Preferred peptidomemetics include those which are azapeptides, those derived from tripeptides, or both. For example, argatroban is a peptidomemetic of L-arginine. Other examples of peptidomemetics for use in the present compositions include, but are not limited to efegatran, inogatran, and napsagatran.

The pharmaceutical compositions of the present invention can be formulated to contain a variety of other chemicals and entities in addition to the active compound, including, but not limited to, one or more of: a pharmaceutically acceptable carrier, a propellant, an excipient, a surfactant, a binding agent, an adjuvant agent (e.g. Albuterol), a flavoring agent or taste masking agent (e.g., sweeteners), a coloring agent, an emulsifying agent, a stabilizing agent (including preservatives, buffers and antioxidants) and/or targeting co-molecules (e.g. liposome entrapped). In many cases, it is preferable to include isotonic agents, for example, sugars, polyalcohols such as mannitol or sorbitol, and sodium chloride in the pharmaceutical composition. For example, in one embodiment, the pharmaceutical composition comprises 25-99.99 percent by weight of fondaparinux, 0-75 percent by weight of an excipient, and 0-3 percent by weight of a surfactant. In another embodiment, for example, the pharmaceutical composition comprises 25-99.99 percent by weight of argatroban, 0-75 percent by weight of an excipient, and 0-3 percent by weight of a surfactant.

The amount of thrombin modulating agent (active ingredient) which can be combined with a carrier material to produce a single dosage form will generally be that amount of the active ingredient which produces a therapeutic effect. In various embodiments, the amount of active ingredient is in the range from about 25 percent to about 99 percent by weight, preferably is in the range from about 95 percent to about 10 percent by weight. It will be appreciated that the compositions of the present invention can be administered in combination with other known pharmaceutical compositions for the treatment of bronchial disorders, either concurrently or sequentially.

Anti-thrombin agents have several properties that make them desirable and effective for the treatment of bronchial inflammation and bronchospasm (See, e.g., Garrigo, Ahmed NEJM, and Ahmed ARCCM 1999). For example, anti-thrombin agents: (1) are generally anti-inflammatory; (2) inhibit mast cell activity including de-granulation; (3) inhibit allergic responses; and (4) decrease neutrophil and other immune cell chemotaxis. (See, e.g., the references Schwartz, Bowler, Matzner, Lucio, Campo, Diamant, Molinari, Ahmed JAP 1993, Ahmed JAP 1994, and Martinez JAP 1998).

Although, a wide variety of compounds have been demonstrated to have direct or indirect ability to inhibit thrombin, they vary enormously in molecular weight and antigenicity. Antigenicity is determined by a variety of factors, including molecular weight (with higher usually being worse), divergence from human equivalent (animal heparin vs. human heparin), class of molecule (proteins are generally more antigenic than carbohydrates), chemical modification (e.g., the glycosylation of proteins determines our blood types, and extensive glycosylation producing more antigenicity), and other factors. It is believed in the present application, without being held to theory, that the likely order of antigenicity of selected thrombin inhibitors (in order of greater to lesser antigenicity) is as follow: lepirudin (animal protein)>unfractionated heparin (animal protein)>low molecular weight heparin (different structure from human heparin)>fondaparinux>argatroban.

In various embodiments, the non-protein thrombin modulating compound comprises one or more of argatroban (chemical name: 1-[5-[(aminoiminomethyl)amino]-1-oxo-2-[[(1,2,3,4-tetrahydro-3-methyl-8-quinolinyl)sulfoyl]amino]pentyl]-4-methyl-2-piperidinecarboxylic acid monohydrate); and fondaparinux (chemical name: methyl O-2-deoxy-6-O-sulfo-2-(sulfoamino)-α-D-glucopyranosyl-((1→4)-O-β-D-glucopyrannuronosyl-(1→4)-O-2-deoxy-3,6-di-O-sulfo-2-(sulfamino)-α-D-glucopyranosyl-(1→4)-O-2-O-sulfo-α-L-iodpyranuronosyl-(1→4)-2-deoxy-6-O-sulfo-2-(sulfamino)-α-D-glucopyranoside).

Argartroban is a synthetic direct thrombin inhibitor. Argartroban is highly selective for thrombin with an inhibitory constant (Kᵢ) of about 0.04 μM. Fondaparinux is an antithrombim III (ATIII)-mediated inhibitor of Factor Xa. Fondaparinux is believed, without being held to theory, to selectively bind to ATIII and potentiate the innate neutralization of Factor Xa by ATIII. Neutralization of Factor Xa interrupts blood coagulation and thus inhibits thrombim formation and thrombus development.

Non-protein thrombin inhibitors (e.g., fondaparinux and argatroban) have several advantages over heparins and other protein based (or protein associated) thrombin inhibitors. First, they are devoid of protein and hence less antigenic. Second, they are sufficiently low in molecular weight that they are unlikely to be antigenic. Third, their low molecular weight makes them chemically and physically more stable, and hence easier to both store and nebulize than proteins.

The quantity of a specific thrombin modulating compound to administer to a patient can be determined by its kinetics, dynamics, and considerations specific to the individual being treated (age, gender, severity of disease, etc), and desired response. Dosages (or molar or pharmacologic equivalents of related compounds to fondaparinux) in the range between about 2.5 micrograms to about 250 milligrams of fondaparinux, in various embodiments of the pharmaceutical composition and methods of the invention, are anticipated to be effective. Dosages of argatroban in the range between about 2 micrograms to about 450 milligrams, in various embodiments of the pharmaceutical composition and methods of the invention, are anticipated to be effective. In various embodiments of the pharmaceutical composition and methods of the invention, particle sizes in the range between about 0.05 microns, to about 25 microns are anticipated to be effective, with the most effective nebulized particles being between about 0.2 microns to about 5 microns in size.

Actual dosage levels of the active ingredients in a pharmaceutical composition of the present invention may be varied so as to obtain an amount of the active ingredient which is effective to achieve the desired therapeutic response for a particular patient, pharmaceutical composition, and mode of administration, without being toxic to the patient. The selected dosage level will depend upon a variety of pharmacokinetic factors including the activity of the particular pharmaceutical compositions of the present invention employed, or the ester, salt or amide thereof, the route of administration, the time of administration, the rate of excretion of the particular compound being employed, the duration of the treatment, other drugs, compounds and/or materials used in combination with the particular pharmaceutical compositions employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could start doses of the active compound employed in a pharmaceutical composition of the invention at levels lower than that required in order to achieve the desired therapeutic effect and gradually increase the dosage until the desired effect is achieved. In general, a suitable dose of a pharmaceutical composition of the invention will be that amount of the compound which is the lowest dose effective to produce a therapeutic effect. Such an effective dose will generally depend upon the factors described above.

Preferably, the pharmaceutical compositions of the present invention comprise one or more additional chemicals and entities, in addition to the thrombin modulating compound, which facilitate, for example, the administration of the active ingredient and treatment of the bronchial disorder. For example, a pharmaceutically acceptable carrier, and/or a propellant can be used to facilitate administration by inhalation therapy (e.g., by atomization, nebulization, aerosolization, dry powderization etc. of at least a portion of the active compound in the formulation). Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Further, for example, an excipient, a surfactant, or both can also be used to further facilitate administration of a thrombin modulating compound. The excipient facilitates the compatibility of the medicament with the propellant and also lowers the discharge pressure to an acceptable range, e.g., from about 2.76×10⁵ to about 5.52×10⁵ newton/meter² absolute (about 40 to about 80 psia), preferably from about 3.45×10⁵ to about 4.83×10⁵ newton/meter² absolute (about 50 to about 70 psia). The excipient chosen must be non-reactive with the medicament, relatively non-toxic, and preferably has a vapor pressure below about 3.45×10⁵ newton/meter² absolute (about 50 psia).

A surfactant can be added to a pharmaceutical composition of the present invention to lower the surface and interfacial tension between the medicament and the propellant. Where the medicament, propellant and excipient are to form a suspension, a surfactant may or may not be required. Where the medicament, propellant and excipient are to form a solution, a surfactant may or may not be necessary, depending in part, on the solubility of the particular medicament and excipient. The surfactant may be any suitable, non-toxic compound which is non-reactive with the medicament and which substantially reduces the surface tension between the medicament, the excipient and the propellant and/or acts as a valve lubricant.

Examples of suitable excipients include, but are not limited to: propylene glycol diesters of medium chain fatty acids; triglyceride esters of medium chain fatty acids, short chains, or long chains, or any combination thereof; perfluorodimothylcyclobutane; perfluorocy clobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

Among the preferred excipients are: propylene glycol diesters of medium chain fatty acids available under the trade name Miglyol 840 (from Huls America, Inc. Piscataway, N.J.); triglyceride esters of medium chain fatty adds available under the trade name Miglyol 812 (from Huls); perfluorodimethylcyclobutane available under the trade name Vertrel 245 (from E. I. DuPont de Nemours and Co., Inc. Wilmington, Del.); perfluorocyclobutane available under the trade name octafluorocyclobutane (from PCR, Gainsville, Fla.); polyethylene glycol available under the trade name PEG 400 (from BASF Parsippany, N.J.); menthol (from Pluess-Stauffer International, Stanford, Conn.); propylene glycol monolaurate available under the trade name lauroglycol (from Gattefosse, Elmsford, N.Y.); diethylene glycol monoethylether available under the trade name Transcutol (from Gattefosse); polyglycolized glyceride of medium chain fatty adds available under the trade name Labrafac Hydro WL 1219 (from Gattefosse); alcohols, such as ethanol, methanol and isopropanol; eucalyptus oil available (from Pluses-Stauffer International); and mixtures thereof.

Examples of suitable surfactants include, but are not limited to: oleic acid; sorbitan trioleate; cetyl pyridinium chloride; soya lecithin; polyoxyethylene(20) sorbitan monolaurate; polyoxyethylene (10) stearyl ether; polyoxyethylene (2) oleyl other; polyoxypropylene-polyoxyethylene ethylene diamine block copolymers; polyoxyethylene(20) sorbitan monostearate; polyoxyethylene(20) sorbitan monooleate; polyoxypropylene-polyoxyethylene block copolymers; castor oil ethoxylate; and combinations thereof.

Among the preferred surfactants are: oleic acid available under the trade name oleic acid NF6321 (from Henkel Corp. Emery Group, Cincinnati, Ohio); cetylpyridinium chloride (from Arrow Chemical, Inc. Westwood, N.J.); soya lecithin available under the trade name Epikuron 200 (from Lucas Meyer Decatur, Ill.); polyoxyethylene(20) sorbitan monolaurate available under the trade name Tween 20 (from ICI Specialty Chemicals, Wilmington, Del.); polyoxyethylene(20) sorbitan monostearate available under the trade name Tween 60 (from ICI); polyoxyethylene(20) sorbitan monooleate available under the trade name Tween 80 (from ICI); polyoxyethylene (10) stearyl ether available under the trade name Brij 76 (from ICI); polyoxyethylene (2) oleyl ether available under the trade name Brij 92 (frown ICI); Polyoxyethylene-polyoxypropylene ethylenediamine block copolymer available under the tradename Tetronic 150 RI (from BASF); polyoxypropylene-polyoxyethylene block copolymers available under the trade names Pluronic L-92, Pluronic L-121 end Pluronic F 68 (from BASF); castor oil ethoxylate available under the trade name Alkasurf CO-40 (from Rhone-Poulenc Mississauga Ontario, Canada); and mixtures thereof. These compounds may be utilized either as the free base, as a salt, or as a clathrate, depending upon the stability and solubility of the active compound in the specific pharmaceutical composition. When clathrates are utilized, P-11 and hexane clathrates are preferred.

In various embodiments, the pharmaceutical compositions of the present invention are formulated for administration by dry powder inhalation and can comprise one or more additional chemicals and entities, in addition to the thrombin modulating compound, which facilitate, for example, the administration of the active ingredient and treatment of the bronchial disorder.

A wide variety of dry-powder inhalers can be used to administer various embodiments of dry powder formulations of the pharmaceutical compositions of the present invention. Examples of commercially available dry-powder inhalers include, but are not limited to, Diskus®, Diskhaler®, and Rotahaler® brand inhalers (GlaxoSmithKline, Inc.), the Turbuhaler™ brand inhaler (AstraZeneca), the HandiHaler® brande inhaler (Boehringer Ingelheim Pharma KG), and the Aerolizer brand inhaler® (Novartis).

Preferably, the pharmaceutical compositions of the present invention comprise one or more additional chemicals and entities, in addition to the thrombin modulating compound, which facilitate, for example, preservation of the sterility and effectiveness of the compositions. For example, in various embodiments, the pharmaceutical composition comprises one or more adjuvants, such as, for example, a preservative, wetting agent, emulsifying agent and/or dispersing agent. Prevention of the presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the pharmaceutical compositions.

In various embodiments, pharmaceutical compositions of the present invention contain an antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may be filled into the aerosol containers using conventional filling equipment. Since propellant 227 may not be compatible with all elastomeric compounds currently utilized in present aerosol valve assemblies, it may be necessary to substitute other materials, such as white buns rubber, or to utilize excipients and optionally surfactants which mitigate the adverse effects of propellant 227 on the valve components.

Depending on the particular application, the container may be charged with a predetermined quantity of pharmaceutical composition for single or multiple dosing. Typically, the container is sized for multiple-dosing, and, therefore, it is very important that the pharmaceutical composition delivered is substantially uniform for each dosing. For example, where the pharmaceutical composition is for bronchodilation, the container typically is charged with a sufficient quantity of the pharmaceutical composition for 200 charges.

Suitable suspensions may be screened in part by observing several physical properties of the pharmaceutical composition, i.e. the rate of particle agglomeration, the size of the agglomerates and the rate of particulate creaming/settling and comparing these to an acceptable standard. Suitable solutions may be screened by observing the solubility of the medicament over the entire recommended storage temperature range. Suspensions of the present invention preferably can be prepared by either the pressure filling or cold filling procedures well-known in the art. For metered dose inhalators, suspensions preferably prepared taking into account efficacy and stability considerations.

Those skilled in the art may choose to add one or more preservatives, buffers, antioxidants, flavors or other taste masking agents (e.g. sweeteners), depending upon the characteristics of the pharmaceutical composition.

Therapeutic pharmaceutical compositions typically must be sterile and stable under the conditions of manufacture and storage. The pharmaceutical composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the pharmaceutical composition.

In various embodiments, equivalents to fondaparinux, argatroban, and mixtures thereof and their equivalents, are intended to be encompassed in the scope of the pharmaceutical compositions and methods of the present invention. In various embodiments, equivalents to compounds such as described in U.S. Pat. No. 6,271.215 to Parish et al., and mixtures thereof and their equivalents are intended to be encompassed in the scope of the pharmaceutical compositions and methods of the present invention.

Various aspects and embodiments of the present invention are further described by way of the following Examples. The Examples are offered by way of illustration and not by way of limitation.

### EXAMPLES

### Example 1

Fondaparinux 2.5 mg in 3 cc saline, pH adjusted with phosphate buffer to a pH in the range of 5.0-8.0, with 2 millimolar EDTA as a preservative, delivered either via a nebulizer or metered dose inhaler 2 times a day for the prevention of bronchial inflammation and bronchospasm.

### Example 2

Fondaparinux, 5 mg, in 3 cc pH adjusted saline (see previous example), 2 mM EDTA preservative, delivered every 4 hours via nebulizer or metered dose inhaler (MDI) as a therapy for acute exacerbation of bronchospasm and/or bronchial irritation.

### Example 3

Fondaparinux 10 mg, liposomal encapsulated (in a liposome that may be engineered to bind to target cells in the airway epithelium such as mast cells and macrophages), 2 mM EDTA as preservative, administered 1 or two times a day by either nebulizer or MDI, as a long-acting prophylactic therapy, or treatment for chronic bronchial inflammation.

### Example 4

Fondaparinux 10 mg, fluticasone 500 ug , salmeterol 50 ug in a liposmal sustained release pharmaceutical composition, containing a surfactant, an excipient, and a taste masking agent.

### Example 5

Fondaparinux, 5 mg in 3 cc pH adjusted with TRIS buffer to a pH of 5.0-8.0 with 2 mM EDTA as a preservative, delivered as a nebulized compound to treat acute bronchial spasm. Dosing interval is shortened in the face of inadequate clinical response to therapy from every 12 hours to every 8 hours, or the dose might be adjusted upward to 7.5 mg or 10 mg (with the same dosing interval).

### Example 6

Fondaparinux 1 mg in propylene glycol esters of medium chain fatty acids, flavored with mint, with a low concentration of ethanol (5%), polyoxyethylene (10) stearyl ether nebulized every 8 hours.

### Example 7

Fondaparinux 100 mg in propylene glycol esters of medium chain fatty acids, with a low concentration of ethanol (3%), polyoxyethylene (10) stearyl ether nebulized every 12 hours.

### Example 8

Argatroban 25 micrograms in 3 cc saline, pH adjusted with phosphate buffer to a pH in the range of 5.0 to 8.0, with 2 millimolar EDTA as a preservative, delivered either via a nebulizer or metered dose inhaler 2 times a day for the prevention and/or treatment of bronchial inflammation and bronchial spasm. cl Exam1ple 9

Argatroban 8000 micrograms, liposome encapsulated (in a liposome engineered to bind to target cells in the airway epithelium such as mast cells, macrophages, and neutrophils), 2 mM EDTA preservative, administered every 4 hours by either nebulizer or MDI, as treatment for bronchial inflammation and bronchial spasm.

### Example 10

Argatroban 280 micrograms, in 3 cc pH adjusted with TRIS buffer to a pH of 5.0-8.0 with 2 mM EDTA as a preservative, administered one time a day as a treatment for bronchial inflammation.

The descriptions of the embodiments and examples of the invention herein have been presented for the purpose of illustration and description. They are not intended to be exhaustive or to limit the invention to the precise forms disclosed. One of ordinary skill in the will realize that many modifications and variations are possible in light of the teachings provided herein. The embodiments and examples herein were chosen and described in order to best explain the principles of the invention and its practical application and to thereby enable others skilled in the art to best utilize the invention in various embodiments and with various modifications as are suited to the particular use contemplated.

The section headings used herein are for organizational purposes only and are not to be construed as limiting the subject matter described in any way.

All publications and patent documents cited herein, including those listed in the REFERENCES section below, are hereby incorporated by reference in their entirety for all purposes and to the same extent as if each were so individually denoted. In the event that one or more of the incorporated literature and similar materials differs from or contradicts this application, including defined terms, term usage, described techniques, or the like, this application controls.

The claims should not be read as limited to the described order or elements unless stated to that effect. It should be understood that various changes in form and detail may be made without departing from the scope of the appended claims. Therefore, all embodiments that come within the scope and spirit of the following claims and equivalents thereto are claimed.

### REFERENCES

1. Tahir Ahmed, Brenda J. Gonzalez, and Ignacio Danta, “Prevention of Exercise-induced Bronchoconstriction by Inhaled Low-molecular-weight Heparin” Am. J. Respir. Crit. Care Med. 160: 576-581;
2. Ahmed T, Garrigo J, Danta I., “Preventing bronchoconstriction in exercise-induced asthma with inhaled heparin.”, N. Engl. J. Med. 329(2):129-30, 1993;
3. J. Martinez-Salas, J. Appl. Physiol., 84: 222, 1998;
4. Molinari J F, Campo C, Shahida S, Ahmed T, “Inhibition of antigen induced airway hyperresponsiveness by ultralow molecular weight heparin”, Am. J. Resp. Crit. Care Med, 157: 887-893, 1998;
5. Campo C, Molinari J F, Ungo J, Ahmed T, “Molecular Weight dependent effects of nonanticoagulant heparins on allergic airway responses”, J. Appl. Physiol., 86:549-57, 1999;
6. Schwartz L B, Bradford T R, “Regulation of tryptase from human lung mast cells by heparin”, J. Biol. Chem., 261: 7372-7379, 1986;
7. Bowler S D, Smith S D, Lavercombe P S, “Heparin inhibits the immediate response to antigen in the skin and lungs of allergic subjects” Am. Rev. Resp. Dis., 147: 160-163, 1993;
8. Ahmed T, Abraham W M, D'Brot J, “Effects of inhaled heparin on immunologic and nonimmunologic bronchoconstrictor responses in sheep”, Am. Rev. Resp. Dis., 145:566-70, 1992;
9. Ahmed T, Garrigo J, Danta I, “Preventing bronchoconstriction in exercise-induced asthma with inhaled heparin”, N. Engl. J. Med., 329:90-95, 1993;
10. Garrigo J, Danta I, Ahmed T, “Time course of the protective effect of inhaled heparin on exercise induced asthma”, Am. J. Resp. Crit. Care Med., 153:102-7, 1996;
11. Diamant Z, Timmers M C, et al., “Effect of inhaled heparin on allergen-induced early and late asthmatic responses in patients with atopic asthma”, Am. J. Resp. Crit. Care Med., 153:1790-5, 1996;
12. Cifonelli J A, “The relationship of molecular weight, and sulfate content and distribution to anticoagulant activity of heparin preparations”, Carbohydrate Research, 7:145-54, 1974;
13. Laurent T C, Tengblad A, Thunberg L, Hook M, Lindahl U, “The molecular-weight dependence of the anti-coagulant activity of heparin”, Biochem. J., 175:691-701, 1978;
14. Ahmed T, Gonzalez B J, Danta I, “Prevention of exercise-induced bronchoconstriction by inhaled low-molecular weight heparin”, Am. J. Resp. Crit. Care Med., 160:576-581, 1999;
15. Matzner Y, Marx G, Drexler R, Eldor A, “The Inhibitory effect of heparin and related glycosaminoglycans on neutrophil chemotaxis”, Thromb. Hemostasis, 52:134-137, 1984;
16. Ahmed T, Syriste T, Lucio J, Abraham W, Robinson M, D'Brot J, “Inhibition of antigen induced airway and cutaneous responses by heparin: a pharmacodynamic study”, J. Appl. Physiol., 74:1492-98, 1993;
17. Ahmed T, Syriste T, Mendelssohn R, Sorace D, Mansour E, Lansing M, Abraham W M, Robinson M J, “Heparin prevents antigen-induced airway hyper-responsiveness:interference with IP3-mediated Mast cell degranulation?”, J. Appl. Physiol., 76:893-901, 1994;
18. Lucio J, D'Brot J, Guo C, Abraham W M, Lichtenstein L M, Kagey-Sobotka A, Ahmed T, “Immunologic mast cell-mediated responses and histamine release are attenuated by heparin”, J. Appl. Physiol., 73: 1093-1101, 1992;
19. Martinez-Salas J, Menedssohn R, Abraham W M, Hsiao B, Ahmed T, “Inhibition of allergic airway responses by inhaled low-molecular weight heparins: molecular weight dependence”, J. Appl. Physiol., 84:222-228, 1998.

We claim:

1. A method for treating a bronchial disorder by the inhalation of one or more compounds which act by inhibiting thrombin directly and indirectly.

2. The method of claim 1 wherein the bronchial disorder is asthma, bronchospasm, bronchial constriction, or bronchial inflammation.

3. The method of claim 1 wherein the one or more compounds are selected from the group comprising fondaparinux, pentasaccharides related to fondaparinux, argatroban, and compounds related to argatroban.

4. The method of claim 1 wherein the one or more compounds are atomized, nebulized, aerosolized, micronized, in the form of a dry powder, or combinations thereof.

5. A method for treating bronchial hypertrophy by the inhalation of one or more compounds which act by inhibiting thrombin and proliferation in bronchial passages.

6. A method for treating a bronchial disorder comprising the step of inhalation of a pharmaceutical composition comprising one or more thrombin modulating compounds.

7. The method of claim 6, wherein the bronchial disorder comprises one or more of asthma, bronchospasm, bronchial constriction, bronchial inflammation, and bronchial hypertrophy.

8. The method of claim 6, wherein one or more of the one or more thrombin modulating compounds is a pentasaccaride.

9. The method of claim 8, wherein one or more of the one or more thrombin modulating compounds is a sulfated pentasaccaride.

10. The pharmaceutical composition of claim 6, wherein one or more of the one or more thrombin modulating compounds is a peptidomemetic.

11. The pharmaceutical composition of claim 10, wherein one or more of the one or more thrombin modulating compounds is a peptidomemetic of a tripeptide.

12. The pharmaceutical composition of claim 10, wherein one or more of the one or more thrombin modulating compounds is a peptidomemetic of an azapeptide.

13. The pharmaceutical composition of claim 6, wherein one or more of the one or more thrombin modulating compounds is selected from the group consisting of fegatran, inogatran, napsagatran, NAPAP, Ro-466240, BM14.1248, L373-102-001u, and ximelagatran.

14. The method of claim 6, wherein one or more of the one or more thrombin modulating compounds is fondaparinux.

15. The method of claim 6, wherein one or more of the one or more thrombin modulating compounds is argatroban.

16. The method of claim 6, wherein the step of inhalation comprises inhalation of an atomized, nebulized, aerosolized, micronized, dry powder, or combinations thereof, form of the pharmaceutical composition.

17. The method of claim 6, wherein the pharmaceutical composition comprises one or more of a pharmaceutically acceptable carrier, a propellant, an excipient, a surfactant, a binding agent, an adjuvant agent, a flavoring agent or taste masking agent, a coloring agent, an emulsifying agent, a stabilizing agent, an isotonic agent, and targeting co-molecules.

18. A pharmaceutical composition formulated for administration by inhalation for use in the treatment of a bronchial disorder, the pharmaceutical composition comprising:
a thrombin modulating agent;
an excipient; and
a surfactant.

19. The pharmaceutical composition of claim 18, wherein the bronchial disorder comprises one or more of asthma, bronchospasm, bronchial constriction, bronchial inflammation, and bronchial hypertrophy.

20. The pharmaceutical composition of claim 18, wherein the pharmaceutical composition is formulated for administration by inhalation by one or more of atomization, nebulization, and aresolization.

21. The pharmaceutical composition of claim 18, wherein one or more of the one or more thrombin modulating compounds is a pentasaccaride.

22. The pharmaceutical composition of claim 18, wherein one or more of the one or more thrombin modulating compounds is a sulfated pentasaccaride.

23. The pharmaceutical composition of claim 18, wherein one or more of the one or more thrombin modulating compounds is a peptidomemetic.

24. The pharmaceutical composition of claim 23, wherein one or more of the one or more thrombin modulating compounds is a peptidomemetic of a tripeptide.

25. The pharmaceutical composition of claim 23, wherein one or more of the one or more thrombin modulating compounds is a peptidomemetic of an azapeptide.

26. The pharmaceutical composition of claim 18, wherein one or more of the one or more thrombin modulating compounds is selected from the group consisting of efegatran, inogatran, napsagatran, NAPAP, Ro-466240, BM14.1248, L373-102-001u, and ximelagatran.

27. The pharmaceutical composition of claim 18, wherein one or more of the one or more thrombin modulating compounds is fondaparinux.

28. The pharmaceutical composition of claim 18, wherein one or more of the one or more thrombin modulating compounds is argatroban.

29. The method of claim 18, wherein the pharmaceutical composition comprises one or more of a pharmaceutically acceptable carrier, a propellant, an excipient, a surfactant, a binding agent, an adjuvant agent, a flavoring agent or taste masking agent, a coloring agent, an emulsifying agent, a stabilizing agent, an isotonic agent, and targeting co-molecules.

30. The method of claim 18, wherein the excipient is selected from the group consisting of propylene glycol diesters of medium chain fatty acids; triglyceride esters of medium chain fatty acids, short chains, or long chains, or any combination thereof; perfluorodimothylcyclobutane; perfluorocy clobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

31. The method of claim 18, wherein the surfactant is selected from the group consisting of oleic acid; sorbitan trioleate; cetyl pyridinium chloride; soya lecithin; polyoxyethylene(20) sorbitan monolaurate; polyoxyethylene (10) stearyl ether; polyoxyethylene (2) oleyl other; polyoxypropylene-polyoxyethylene ethylene diamine block copolymers; polyoxyethylene(20) sorbitan monostearate; polyoxyethylene(20) sorbitan monooleate; polyoxypropylene-polyoxyethylene block copolymers; castor oil ethoxylate; and combinations thereof.
